(19) United States
(12) Patent Application Publication
(10) Pub. No.: US 2025/0009310 A1
(43) Pub. Date: Jan. 9, 2025

(54) Computer Implemented Method for Determining a Medical Parameter, Training Method and System

(71) Applicant: BIOTRONIK SE & Co. KG, Berlin (DE)

(72) Inventors: Bjoern Henrik DIEM, Berlin (DE); Antje LINNEMANN, Berlin (DE); Anastasia REICH, Berlin (DE)

(73) Assignee: BIOTRONIK SE & Co. KG, Berlin (DE)

(21) Appl. No.: 18/697,924

(22) PCT Filed: Sep. 20, 2022
(86) PCT No.: PCT/EP2022/076014

§ 371 Date: Apr. 2, 2024

(30) Foreign Application Priority Data
Oct. 4, 2021 (EP) 21200657.1

(51) Int. Cl. A61B 5/00; A61B 5/02; A61B 5/024; A61B 5/0537; A61B 5/0538; A61B 5/11; A61B 5/29; A61B 5/346

(52) U.S. Cl. CPC A61B 5/7267; A61B 5/02028; A61B 5/024; A61B 5/0537; A61B 5/0538; A61B 5/11; A61B 5/29; A61B 5/346; A61B 5/686; A61B 5/7275; A61B 2560/045

(57) ABSTRACT

A computer implemented method for determining an ejection fraction, comprising the steps of receiving a first data set comprising pre-acquired cardiac current curve data, in particular one-channel cardiac current curve data, captured by an implantable medical device, applying a machine learning algorithm to the pre-acquired cardiac current curve data, and outputting a second data set representing the ejection fraction and/or the variation of the ejection fraction or a classification of the ejection fraction and/or a classification of the variation of the ejection fraction by the machine learning algorithm. Furthermore, a corresponding system and a method for providing a trained machine learning algorithm is provided.

### CROSS-REFERENCE TO RELATED APPLICATIONS

This application is the United States National Phase under 35 U.S.C. § 371 of PCT International Patent Application No. PCT/EP2022/076014, filed on Sep. 20, 2022, which claims the benefit of European Patent Application No. 21200657.1, filed on Oct. 4, 2021, the disclosures of which are hereby incorporated by reference herein in their entireties.

### TECHNICAL FIELD

The present invention relates to a computer implemented method for determining an ejection fraction and/or a variation of the ejection fraction or a classification of the ejection fraction and/or a classification of the variation of the ejection fraction.

### BACKGROUND

Furthermore, the present invention relates to a computer implemented method for providing a trained machine learning algorithm configured to determine an ejection fraction and/or a variation of the ejection fraction or a classification of the ejection fraction and/or a classification of the variation of the ejection fraction.

In addition, the present invention relates to a system for determining an ejection fraction and/or a variation of the ejection fraction or a classification of the ejection fraction and/or a classification of the variation of the ejection fraction.

Cardiac insufficiency or heart failure patients require regular monitoring of their cardiac pump function. This is measured by imaging methods as ejection fraction. However, this requires the patient's presence at the physician's practice. The imaging procedures, e.g., a cardiac echo, an MRI of the heart, a CT of the heart, or a catheter examination of the heart, involve relevant time and technical/personal effort and, in some cases, risk for the patient. Remote transmission of a 12-lead ECG further requires the active cooperation and compliance of the patient, who may be overtaxed.

U.S. Publication No. 2020/0046286 A1 discloses methods and techniques, both non-invasive and invasive, for characterizing cardiovascular systems from single channel biological data such as electrocardiogramarfusion, bioimpedance and pressure waves. More specifically, the disclosure relates to methods that utilize data to identify targets with clinical, pharmacological, or basic research utility such as, but not limited to, disease states, cardiac structural defects, functional cardiac deficiencies induced by teratogens and other toxic agents, pathological substrates, conduction delays and defects, and ejection fraction. The single channel data can be obtained from devices such as a single channel recorder, implantable telemeter, smartphone or other smart handheld consumer device, smart watch, perfusion sensor, clothing embedded with biometrics sensors, devices that utilize two hands for data collection, and other similar data sources.

The present disclosure is directed toward overcoming one or more of the above-mentioned problems, though not necessarily limited to embodiments that do.

### SUMMARY

It is therefore an object of the present invention to provide an improved method for early detection of new onset of heart failure in patients with active cardiac implants and without known heart failure.

At least the object is solved by a computer implemented method for determining an ejection fraction and/or a variation of the ejection fraction or a classification of the ejection fraction and/or a classification of the variation of the ejection fraction having the features of claim 1.

Furthermore, at least the object is solved by a computer implemented method for providing a trained machine learning algorithm configured to determine an ejection fraction and/or a variation of the ejection fraction or a classification of the ejection fraction and/or a classification of the variation of the ejection fraction having the features of claim 14.

Moreover, at least the object is solved by a system for determining an ejection fraction and/or a variation of the ejection fraction or a classification of the ejection fraction and/or a classification of the variation of the ejection fraction having the features of claim 15.

Further developments and advantageous embodiments are defined in the dependent claims.

The present invention provides a computer implemented method for determining an ejection fraction and/or a variation of the ejection fraction or a classification of the ejection fraction and/or a classification of the variation of the ejection fraction.

The method comprises receiving a first data set comprising pre-acquired cardiac current curve data, in particular one-channel cardiac current curve data, captured by an implantable medical device.

Furthermore, the method comprises applying a machine learning algorithm to the pre-acquired cardiac current curve data, and outputting a second data set representing the ejection fraction and/or the variation of the ejection fraction or a classification of the ejection fraction and/or a classification of the variation of the ejection fraction by the machine learning algorithm.

The present invention further provides a computer implemented method for providing a trained machine learning algorithm configured to determine an ejection fraction and/or a variation of the ejection fraction or a classification of the ejection fraction and/or a classification of the variation of the ejection fraction.

The method comprises receiving a first training data set comprising pre-acquired cardiac current curve data, in particular one-channel cardiac current curve data, captured by an implantable medical device.

Moreover, the method comprises receiving a second training data set representing an ejection fraction and/or a variation of the ejection fraction or a classification of the ejection fraction and/or a classification of the variation of the ejection fraction.

In addition, the method comprises training the machine learning algorithm by an optimization algorithm which calculates an extreme value of a loss function for regression of the ejection fraction and/or the variation of the ejection fraction from the pre-acquired cardiac current curve data or for classification of the ejection fraction and/or classification of a variation of the ejection fraction from the pre-acquired cardiac current curve data.

The present invention further provides a system for determining an ejection fraction and/or a variation of the ejection fraction or a classification of the ejection fraction and/or a classification of the variation of the ejection fraction.

The system comprises means for receiving a first data set comprising pre-acquired cardiac current curve data, in particular one-channel cardiac current curve data, captured by an implantable medical device and means for applying a machine learning algorithm to the pre-acquired cardiac current curve data.

Furthermore, the system comprises means for outputting a second data set representing the ejection fraction and/or the variation of the ejection fraction or a classification of the ejection fraction and/or a classification of the variation of the ejection fraction.

An idea of the present invention is to provide automated remote monitoring of an ejection fraction with higher frequency than possible by outpatient follow-up. As a result, an improvement of therapy quality with early detection of the need for patient-specific therapy change can be achieved.

The implantable medical device such as an implantable cardiac pacemaker or an implantable cardioverter-defibrillator regularly records cardiac current curve data and transmits this via a patient device to a central server. There, preferably the ejection fraction is determined from the transmitted data using the machine learning algorithm.

If a value of the ejection fraction lies outside the limits set individually for the patient by the physician or if there are changes to the previously transmitted values, the physician is automatically informed via a suitable medium such as e-mail. Thus, an improvement of therapy quality can be achieved.

The machine learning algorithm such as an artificial neural net thus advantageously is able to accurately determine the ejection fraction and/or the variation of the ejection fraction or classify the ejection fraction and/or a classify the variation of the ejection fraction based on solely a one-channel cardiac current curve.

The cardiac current curve can be, e.g., a subcutaneous ECG, a pseudo-ECG between a shock coil and the implantable medical device or intracardiac current waveforms.

The ejection fraction is the volumetric fraction or portion of the total of fluid ejected from a chamber, usually the heart, with each contraction or heartbeat.

Machine learning algorithms are based on using statistical techniques to train a data processing system to perform a specific task without being explicitly programmed to do so. The goal of machine learning is to construct algorithms that can learn from data and make predictions. These algorithms create mathematical models that can be used, for example, to classify data or to solve regression type problems.

According to an aspect of the present invention, if the second data set represents the variation of the ejection fraction, the second data set is used to calculate an absolute value of the ejection fraction. The variation of the ejection fraction determined by the machine learning algorithm can thus advantageously be used to calculate the absolute value of the ejection fraction by statistical methods.

According to a further aspect of the present invention, the machine learning algorithm is a regression-type algorithm, wherein the second data set is given by at least one numeric value, in particular a sequence of numeric values, representing the ejection fraction and/or the variation of the ejection fraction. The machine learning algorithm can thus advantageously either be trained to predict the ejection fraction and/or the variation of the ejection fraction.

According to a further aspect of the present invention, the machine learning algorithm is a classification-type algorithm, wherein the second data set comprises at least one of a first class representing the ejection fraction and/or the variation of the ejection fraction of a normal patient condition and a second class representing the ejection fraction and/or the variation of the ejection fraction of an abnormal patient condition.

This provides the advantage that as opposed to the regression-type algorithm a further step of determining whether or not the predicted ejection fraction and/or the variation of the ejection fraction is within a predetermined range or does not exceed a predetermined threshold value can be omitted since this evaluation is already comprised in the respective classification result.

According to a further aspect of the present invention, the second data set further comprises a third class representing that the classification of the ejection fraction and/or the classification of the variation of the ejection fraction is indeterminable from the first data set, in particular from a specific heartbeat of the pre-acquired cardiac current curve data.

Should the determined classification represent that the classification of the ejection fraction and/or the classification of the variation of the ejection fraction is indeterminable from the first data set the result can be discarded, i.e., no notification will be sent to the healthcare provider.

According to a further aspect of the present invention, if at least one value of the second data set representing the ejection fraction and/or the variation of the ejection fraction is outside a predetermined numeric range or is above or below a predetermined threshold value, in particular if the at least one value is outside limits set individually for a patient by a physician and/or if the at least one value differs by a predetermined amount from previously transmitted values, a notification is sent to a communication device of a health care provider.

The healthcare provider is thus advantageously informed about an early detection of new onset of heart failure in patients with active cardiac implants and without known heart failure compared to conventional aftercare visits of the patient.

According to a further aspect of the present invention, if the machine learning algorithm classifies the ejection fraction of an abnormal patient condition and/or the variation of the ejection fraction of an abnormal patient condition, a notification is sent to a communication device of a health care provider.

According to a further aspect of the present invention, the at least one value of the second data set representing the ejection fraction and/or the variation of the ejection fraction is evaluated by performing a trend analysis of at least one further value of the second data set representing the ejection fraction and/or the variation of the ejection fraction, wherein if the trend analysis meets predetermined criteria of an abnormal patient condition, a notification is sent to a communication device of a health care provider.

The healthcare provider is thus advantageously informed about an early detection of new onset of heart failure in patients with active cardiac implants and without known heart failure compared to conventional aftercare visits of the patient.

According to a further aspect of the present invention, a reference value of the ejection fraction and/or the variation of the ejection fraction is compared to the second data set outputted by the machine learning algorithm representing the ejection fraction and/or a variation of the ejection fraction to calibrate the output of the machine learning algorithm. The reference value of the ejection fraction and/or the variation of the ejection fraction may be obtained, among other things, by a twelve-channel ECG, an echo or a magnetic resonance tomography (MRT).

Based on the reference value of the ejection fraction and/or the variation of the ejection fraction, a most appropriate machine learning algorithm is selected from a library of machine learning algorithms. In this case, and appropriate machine learning algorithm from available multiple machine learning algorithms that matches the individual patient's reference value can be selected.

According to a further aspect of the present invention, based on the reference value of the ejection fraction and/or the variation of the ejection fraction, a most appropriate machine learning algorithm is selected from a library of machine learning algorithms.

In this case, an appropriate machine learning algorithm from available multiple machine learning algorithms that matches the individual patient's reference value can be selected.

According to a further aspect of the present invention, the first data set further comprises a heart rate, a thorax impedance and/or a patient activity captured by an implantable medical device. In using said further medical parameters the machine learning algorithm can advantageously generate more accurate results of the output data.

According to a further aspect of the present invention, the cardiac current curve data is acquired by the implantable medical device at predetermined intervals and/or on request, in particular as a wide-field ECG between electrodes and a housing of the implantable medical device, and wherein the cardiac current curve data is transmitted to a central server via a patient communication device or smartphone.

It is therefore advantageously not necessary for the patient to perform, for example, a multi-channel ECG, echo, MRT, etc. in a clinical setting. Furthermore, the output data of the algorithm can thus be transmitted to the server for further evaluation according to the predetermined intervals and/or on request thus significantly shortening the time to potentially detect new onset of heart failure in patients with active cardiac implants and without known heart failure.

According to a further aspect of the present invention, the first data set comprises a first cardiac current curve recorded by the implantable medical device at a first time interval and a second cardiac current curve recorded by the implantable medical device at a second time interval, in particular offset from the first time interval, and wherein the machine learning algorithm is configured to determine the variation in the ejection fraction from the variation between the first cardiac current curve and the second cardiac current curve.

Thus advantageously, the variation in the ejection fraction can be determined from the variation data of the respective cardiac current curves.

The herein described features of the implantable system for determining an ejection fraction and/or a variation of the ejection fraction or a classification of the ejection fraction and/or a classification of the variation of the ejection fraction are also disclosed for the computer implemented method for determining an ejection fraction and/or a variation of the ejection fraction or a classification of the ejection fraction and/or a classification of the variation of the ejection fraction and vice versa.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

For a more complete understanding of the present invention and advantages thereof, reference is now made to the following description taken in conjunction with the accompanying drawings. The present invention is explained in more detail below using exemplary embodiments, which are specified in the schematic figures of the drawings, in which:

FIG. 1 shows a flowchart of a computer implemented method and system for determining an ejection fraction and/or a variation of the ejection fraction or a classification of the ejection fraction and/or a classification of the variation of the ejection fraction according to a preferred embodiment of the present invention; and

FIG. 2 shows a flowchart of a computer implemented method for providing a trained machine learning algorithm configured to determine an ejection fraction and/or a variation of the ejection fraction or a classification of the ejection fraction and/or a classification of the variation of the ejection fraction according to the preferred embodiment of the present invention.

### DETAILED DESCRIPTION

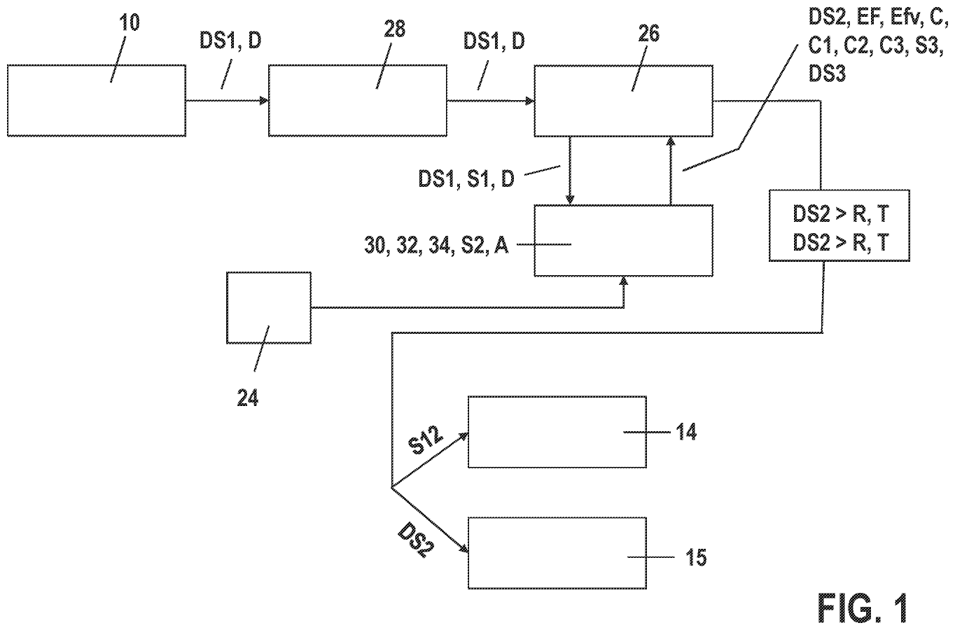

The system shown in FIG. 1 for determining an ejection fraction EF and/or a variation of the ejection fraction EFv or a classification C of the ejection fraction EF and/or a classification C of the variation of the ejection fraction EFv comprises means 30 for receiving S1 a first data set DS1 comprising pre-acquired cardiac current curve data D, in particular one-channel cardiac current curve data D, captured by an implantable medical device 10.

Furthermore, the system comprises means 32 for applying S2 a machine learning algorithm A to the pre-acquired cardiac current curve data D, and means 34 for outputting S3 a second data set DS2 representing the ejection fraction EF and/or the variation of the ejection fraction EFv or a classification C of the ejection fraction EF and/or a classification C of the variation of the ejection fraction EFv.

If the second data set DS2 represents the variation of the ejection fraction EFv, the second data set DS2 is used to calculate an absolute value of the ejection fraction EF.

The machine learning algorithm A is preferably a regression-type algorithm, wherein the second data set DS2 is given by at least one numeric value, in particular a sequence of numeric values, representing the ejection fraction EF and/or the variation of the ejection fraction EFv

Alternatively, the machine learning algorithm A can be a classification-type algorithm, wherein the second data set DS2 comprises at least one of a first class C1 representing the ejection fraction EF and/or the variation of the ejection fraction EFv of a normal patient condition and a second class C2 representing the ejection fraction EF and/or the variation of the ejection fraction EFv of an abnormal patient condition.

The second data set DS2 further comprises a third class C3 representing that the classification of the ejection fraction EF and/or the classification of the variation of the ejection fraction EFv is indeterminable from the first data set DS1, in particular from a specific heartbeat of the pre-acquired cardiac current curve data D.

If at least one value of the second data set DS2 representing the ejection fraction EF and/or the variation of the ejection fraction EFv is outside a predetermined numeric range R or is above or below a predetermined threshold value V, in particular if the at least one value is outside limits set individually for a patient by a physician and/or if the at least one value differs by a predetermined amount from previously transmitted values, a notification 12 is sent to a communication device 14 of a health care provider.

If the machine learning algorithm A classifies the ejection fraction EF of an abnormal patient condition and/or the variation of the ejection fraction EFv of an abnormal patient condition, a notification 12 is sent to a communication device 14 of a health care provider.

The at least one value of the second data set DS2 representing the ejection fraction EF and/or the variation of the ejection fraction EFv is evaluated by performing a trend analysis of at least one further value of the second data set DS2 representing the ejection fraction EF and/or the variation of the ejection fraction EFv. Furthermore, if the trend analysis meets predetermined criteria of an abnormal patient condition, a notification 12 is sent to a communication device 14 of a health care provider.

Said notification 12 is preferably sent by e-mail. Alternatively, the notification 12 may be sent by text message (SMS) or by means of an in-app notification. Furthermore, the healthcare provider may access the at least one value of the second data set DS2 representing the ejection fraction EF and/or the variation of the ejection fraction EFv via a front-end application 15 on a suitable communication device such as a smart phone and/or a personal computer.

A reference value 24 of the ejection fraction EF and/or the variation of the ejection fraction EFv is compared to the second data set DS2 outputted by the machine learning algorithm A representing the ejection fraction EF and/or a variation of the ejection fraction EFv to calibrate the output of the machine learning algorithm A. The reference value 24 of the ejection fraction and/or the variation of the ejection fraction may be obtained by a twelve-channel ECG, an echo or an MRT.

Based on the reference value 24 of the ejection fraction EF and/or the variation of the ejection fraction EFv, a most appropriate machine learning algorithm A is selected from a library of machine learning algorithms A.

The first data set DS1 further comprises a heart rate, a thorax impedance and/or a patient activity captured by an implantable medical device 10.

The cardiac current curve data D is acquired by the implantable medical device 10 at predetermined intervals and/or on request, in particular as a wide-field ECG between electrodes and a housing of the implantable medical device 10. The cardiac current curve data D is transmitted to a central server 26 via a patient communication device 28 or smartphone.

The first data set DS1 comprises a first cardiac current curve recorded by the implantable medical device 10 at a first time interval and a second cardiac current curve recorded by the implantable medical device 10 at a second time interval, in particular offset from the first time interval, and wherein the machine learning algorithm A is configured to determine the variation in the ejection fraction EF from the variation between the first cardiac current curve and the second cardiac current curve.

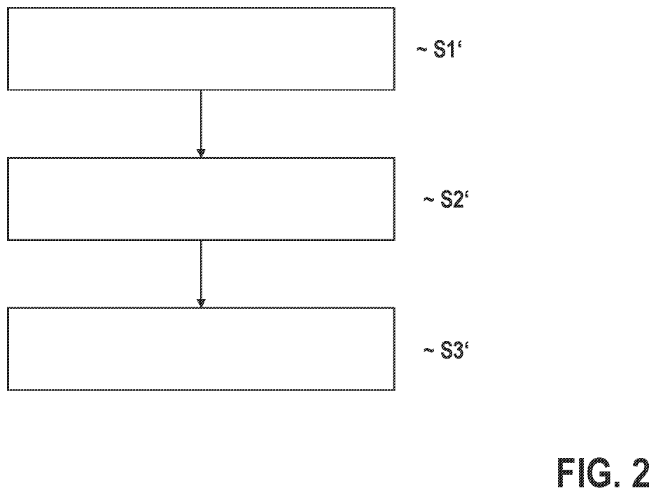

FIG. 2 shows a flowchart of a computer implemented method for providing a trained machine learning algorithm configured to determine an ejection fraction EF and/or a variation of the ejection fraction EFv or a classification C of the ejection fraction EF and/or a classification C of the variation of the ejection fraction EFv according to the preferred embodiment of the present invention.

The method comprises receiving S1′ a first training data set comprising pre-acquired cardiac current curve data D, in particular one-channel cardiac current curve data D, captured by an implantable medical device 10.

Furthermore, the method comprises receiving S2′ a second training data set representing an ejection fraction EF and/or a variation of the ejection fraction EFv or a classification C of the ejection fraction EF and/or a classification C of the variation of the ejection fraction EFv.

In addition, the method comprises training S3′ the machine learning algorithm A by an optimization algorithm which calculates an extreme value of a loss function for regression of the ejection fraction EF and/or the variation of the ejection fraction EFv from the pre-acquired cardiac current curve data D or for classification C of the ejection fraction EF and/or classification C of a variation of the ejection fraction EFv from the pre-acquired cardiac current curve data D.

The machine learning algorithm A configured to determine an ejection fraction and/or a variation of the ejection fraction or a classification of the ejection fraction and/or a classification of the variation of the ejection fraction is trained using corresponding pairs of the first training data set and the second training data set.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

### REFERENCE SIGNS

10 implantable medical device
12 notification
14 communication device
15 front-end application
24 reference value
26 central server
28 patient communication device
30 means
32 means
34 means
A machine learning algorithm
C classification
C1 first class
C2 second class
C3 third class
D cardiac current curve data
DS1 first data set
DS2 second data set
EF ejection fraction
EFv variation of ejection fraction
R predetermined numeric range
S1-S3 method steps
S1′-S3′ method steps
V threshold value

1. Computer implemented method for determining an ejection fraction and/or a variation of the ejection fraction or a classification of the ejection fraction and/or a classification of the variation of the ejection fraction, comprising the steps of:
receiving a first data set comprising pre-acquired cardiac current curve data, in particular one-channel cardiac current curve data, captured by an implantable medical device;
applying a machine learning algorithm to the pre-acquired cardiac current curve data; and
outputting a second data set representing the ejection fraction and/or the variation of the ejection fraction or a classification of the ejection fraction and/or a classification of the variation of the ejection fraction by the machine learning algorithm.

2. Computer implemented method of claim 1, wherein if the second data set represents the variation of the ejection fraction, the second data set is used to calculate an absolute value of the ejection fraction.

3. Computer implemented method of claim 1, wherein the machine learning algorithm is a regression-type algorithm, wherein the second data set is given by at least one numeric value, in particular a sequence of numeric values, representing the ejection fraction and/or the variation of the ejection fraction.

4. Computer implemented method of claim 1, wherein the machine learning algorithm is a classification-type algorithm, wherein the second data set comprises at least one of a first class representing the ejection fraction and/or the variation of the ejection fraction of a normal patient condition and a second class representing the ejection fraction and/or the variation of the ejection fraction of an abnormal patient condition.

5. Computer implemented method of claim 4, wherein the second data set further comprises a third class representing that the classification of the ejection fraction and/or the classification of the variation of the ejection fraction is indeterminable from the first data set, in particular from a specific heartbeat of the pre-acquired cardiac current curve data.

6. Computer implemented method of claim 1, wherein if at least one value of the second data set representing the ejection fraction and/or the variation of the ejection fraction is outside a predetermined numeric range or is above or below a predetermined threshold value, in particular if the at least one value is outside limits set individually for a patient by a physician and/or if the at least one value differs by a predetermined amount from previously transmitted values, a notification is sent to a communication device of a health care provider.

7. Computer implemented method of claim 1, wherein if the machine learning algorithm classifies the ejection fraction of an abnormal patient condition and/or the variation of the ejection fraction of an abnormal patient condition, a notification is sent to a communication device of a health care provider.

8. Computer implemented method of claim 1, wherein the at least one value of the second data set representing the ejection fraction and/or the variation of the ejection fraction is evaluated by performing a trend analysis of at least one further value of the second data set representing the ejection fraction and/or the variation of the ejection fraction, wherein if the trend analysis meets predetermined criteria of an abnormal patient condition, a notification is sent to a communication device of a health care provider.

9. Computer implemented method of claim 1, wherein a reference value of the ejection fraction and/or the variation of the ejection fraction is compared to the second data set outputted by the machine learning algorithm representing the ejection fraction and/or a variation of the ejection fraction to calibrate the output of the machine learning algorithm.

10. Computer implemented method of claim 9, wherein based on the reference value of the ejection fraction and/or the variation of the ejection fraction, a most appropriate machine learning algorithm is selected from a library of machine learning algorithms.

11. Computer implemented method of claim 1, wherein the first data set further comprises a heart rate, a thorax impedance and/or a patient activity captured by an implantable medical device.

12. Computer implemented method of claim 1, wherein the cardiac current curve data is acquired by the implantable medical device at predetermined intervals and/or on request, in particular as a wide-field ECG between electrodes and a housing of the implantable medical device, and wherein the cardiac current curve data is transmitted to a central server via a patient communication device or smartphone.

13. Computer implemented method of claim 1, wherein the first data set comprises a first cardiac current curve recorded by the implantable medical device at a first time interval and a second cardiac current curve recorded by the implantable medical device at a second time interval, in particular offset from the first time interval, and wherein the machine learning algorithm is configured to determine the variation in the ejection fraction from the variation between the first cardiac current curve and the second cardiac current curve.

14. Computer implemented method for providing a trained machine learning algorithm configured to determine an ejection fraction and/or a variation of the ejection fraction or a classification of the ejection fraction and/or a classification of the variation of the ejection fraction, comprising the steps of:
receiving a first training data set comprising pre-acquired cardiac current curve data, in particular one-channel cardiac current curve data, captured by an implantable medical device;
receiving a second training data set representing an ejection fraction and/or a variation of the ejection fraction or a classification of the ejection fraction and/or a classification of the variation of the ejection fraction; and
training the machine learning algorithm by an optimization algorithm which calculates an extreme value of a loss function for regression of the ejection fraction and/or the variation of the ejection fraction from the pre-acquired cardiac current curve data or for classification of the ejection fraction and/or classification of a variation of the ejection fraction from the pre-acquired cardiac current curve data.

15. System for determining an ejection fraction and/or a variation of the ejection fraction or a classification of the ejection fraction and/or a classification of the variation of the ejection fraction, comprising:
means for receiving a first data set comprising pre-acquired cardiac current curve data, in particular one-channel cardiac current curve data, captured by an implantable medical device;
means for applying a machine learning algorithm to the pre-acquired cardiac current curve data; and
means for outputting a second data set representing the ejection fraction and/or the variation of the ejection fraction or a classification of the ejection fraction and/or a classification of the variation of the ejection fraction.
